(19) United States
(12) Patent Application Publication
(10) Pub. No.: US 2022/0184278 A1
(43) Pub. Date: Jun. 16, 2022

(54) BICOMPONENT FIBER-BASED SCAFFOLDS FOR MULTIPLE TISSUE JUNCTION REGENERATION

(71) Applicants: North Carolina State University, Raleigh, NC (US); SP Innovations, LLC, Raleigh, NC (US)

(72) Inventors: Martin W. King, Raleigh, NC (US); Edwin Cadet, Raleigh, NC (US); Yihan Huang, Raleigh, NC (US); Yu Xie, Raleigh, NC (US); Harshini Ramakrishna, Tewksbury, MA (US)

(21) Appl. No.: 17/549,056

(22) Filed: Dec. 13, 2021

Related U.S. Application Data
(60) Provisional application No. 63/124,324, filed on Dec. 11, 2020.

(51) Int. Cl. A61L 27/36; A61L 27/18; A61L 27/24; A61L 27/58

(52) U.S. Cl. CPC A61L 27/3662; A61L 27/18; A61L 2430/10; A61L 27/58; A61L 27/24

(57) ABSTRACT

The present invention provides a fiber-based scaffold constructed with collagen fibers and resorbable synthetic fibers for multiple tissue joint regeneration. The scaffold provides both superior biological performance to encourage cellular infiltration and healing and excellent mechanical properties, similar to native tendon tissue. The invention further provides a method for making the scaffold and a method for attaching the scaffold to a host. An exemplary use of the scaffold is in rotator cuff repair or augmentation.

### CROSS-REFERENCE

This application claims priority to U.S. Provisional Application Ser. No. 63/124,324, filed on Dec. 11, 2020, which is incorporated herein by reference in its entirety.

### FIELD

The present invention relates generally to fiber-based scaffolds comprising a biological component to promote tenocyte proliferation and an absorbable synthetic component to enhance mechanical properties to promote multiple tissue junction regeneration. An example is a bicomponent scaffold applicable to rotator cuff tendon repair.

### BACKGROUND

There are more than 28 million people who suffer from musculoskeletal injuries every year in the United States with an associated cost of more than $254 billion. Within this cohort a rotator cuff injury is the second most common injury with an estimated 75,000 repair procedures performed annually. The rotator cuff serves important functions in the connection between the humerus and the scapula and, as a dynamic stabilizer of the glenohumeral joint, it also allows for the rotation of the shoulder joint about its longitudinal axis.

Rotator cuff tendons are the most frequently injured tendons. Surgical reports showed that 16% of the general population suffer from rotator cuff injuries, and the incidence increases to 21% for elderly people, and jumps dramatically to 80% for people older than 80 years. 27% and 37% of the general population are affected by full and partial rotator cuff tears respectively, and they are associated with high recurrence rates after surgical repair. Relatively high failure rates have been reported, particularly after the treatment of large and massive rotator cuff tears, where the risk of recurrence after 1-year is as high as 94%. Tissue quality is one of the crucial factors that correlates closely with repair failure. With poor quality tendon tissue, a scaffold or synthetic augmentation device is considered an effective way to reinforce the repair site and provide biomechanical support that can shield the repaired rotator cuff tendon from excessive applied stress.

An ideal scaffold for rotator cuff repair should have both excellent mechanical properties, similar to native tendon tissue, as well as a superior biological performance to encourage cellular infiltration and healing. Mechanical support is needed, not only during the initial post-operative period, but also during the extended follow-up period when new tissue is being regenerated and remodeled. By providing dimensional stability and mechanical integrity, the healing rate will be more rapid and improved tissue quality will be generated.

The use of either a collagen or extracellular matrix (ECM) graft or a synthetic augmentation device have been widely used for the repair of an injured rotator cuff so as to reinforce the torn tendon tissue. However, both biological ECM and synthetic materials have their advantages and disadvantages. ECM grafts have superior biocompatibility but are unable to provide an equivalent level of mechanical support as synthetic materials.

There is a need for scaffolds that provide both mechanical support and graft/host integration during the healing of the repaired tendon/bone junction. There is a need for extracellular matrix materials that have improved mechanical properties. There is a need for the mechanical support to remain for a period of time commensurate with the healing process (i.e., a need for the scaffold to augment mechanical support until healing allows the affected musculoskeletal system to do so).

There is a need to be able to fabricate scaffolds in a range of different shapes, sizes and thickness on commercial production weaving and braiding equipment to provide versatile textile or fiber-based scaffolds at a comparatively low cost. There is a need for fiber-based scaffolds to be constructed of fibers that are strong enough under dry conditions to allow for production in commercial textile machinery as well as strong enough under wet conditions to provide mechanical stability of the musculoskeletal repair.

### BRIEF SUMMARY OF THE INVENTION

According to an embodiment of the present invention, a fiber-based scaffold for multiple tissue junction regeneration comprises biological collagen fibers and resorbable synthetic fibers. When the scaffold is attached to a musculoskeletal system in a host, the synthetic fibers remain in the host for a period ranging from 6 weeks to 3 years.

According to another embodiment of the present invention, a method for making a fiber-based scaffold for multiple tissue junction regeneration comprises: a) obtaining biological collagen fibers, b) obtaining resorbable synthetic fibers, and c) fabricating the collagen fibers and the synthetic fibers into the scaffold. The scaffold comprises the biological collagen fibers and the resorbable synthetic fibers. When the scaffold is attached to a musculoskeletal system in a host, the synthetic fibers remain in the host for a period ranging from 6 weeks to 3 years.

According to yet another embodiment of the present invention, a method for attaching a fiber-based scaffold for multiple tissue junction regeneration to a host comprises: a) obtaining the scaffold, b) securing a first section of the scaffold to a first site of the musculoskeletal system of the host; and c) securing a second section of the scaffold to a second site of the musculoskeletal system of the host, wherein the second section is distal from the first section. The first site and the second site are joined in tension by the scaffold. The scaffold comprises biological collagen fibers and resorbable synthetic fibers. When the scaffold is attached to the musculoskeletal system in the host, the synthetic fibers remain in the host for a period ranging from 6 weeks to 3 years.

### BRIEF DESCRIPTION OF THE DRAWINGS

The present invention is illustrated and described herein with reference to the various drawings, in which like reference numbers denote like method steps and/or system components, respectively, and in which:

FIG. 1 is a schematic of an anterior view and a posterior view of rotator cuff muscles;

FIG. 2 is a schematic of an anterior view of two illustrative rotator cuff tears;

FIG. 3 is a schematic of a coronal view of a nonlimiting embodiment of the present invention used in a Trans-osseous equivalent suture repair;

FIG. 4 is a graphical illustration of the percent of collagen in a tendon, on a tendon dry weight basis, and the percent of type I and type III and other collagens on a total collagen basis;

FIG. 5 is a schematic of a perspective view of an illustrative electrochemical alignment of collagen process for making collagen fiber;

FIG. 6 is a microscopic image of tenocytes used to evaluate the biocompatibility of the scaffolds;

FIG. 7 is a perspective view of a 16 ends Steeger circular braider;

FIG. 8 is a perspective view of a Instron Model 5584 mechanical tester;

FIG. 9 is a graphical illustration of tensile test results showing maximum load, yield load, and load at 5 mm elongation for wet and dry COL/PLA scaffolds (Ex. 1) and wet and dry PLA scaffolds (Comp. Ex. 1);

FIG. 10 is a graphical illustration of tensile test results showing the maximum stress for wet and dry COL/PLA scaffolds (Ex. 1) and wet and dry PLA scaffolds (Comp. Ex. 1) as well as the upper and lower limits for native rotator cuff tendons;

FIG. 11 is a graphical illustration of tensile test results showing the elongation at maximum load for wet and dry COL/PLA scaffolds (Ex. 1) and wet and dry PLA scaffolds (Comp. Ex 1) as well as the upper limit for native rotator cuff tendons;

FIG. 12 is a graphical illustration of tensile test results showing the elastic modulus for wet and dry COL/PLA scaffolds (EX. 1) and wet and dry PLA scaffolds (Comp. Ex. 1) as well as the upper and lower limits for native rotator cuff tendons;

FIG. 13a is a plan view of rat tenocytes cell cultures in a 24-well plate with each well containing 1 cm×cm COL/PLA scaffolds or PLA scaffolds and 1×10⁴ tenocytes in a media, the rows marked RC contain rotator cuff tenocytes (Ex. 2 and Comp. Ex 2) and the rows marked AC contain acromioclavicular join tenocytes (Ex. 3 and Comp. Ex. 3);

FIG. 13b is a plan view of samples of the media from the COL/PLA and PLA scaffolds seeded with the rat rotator cuff tenocytes (Ex. 2 and Comp. Ex. 2) tested using 10% ALARMARBLUE at 1, 7, 14, and 28 days, as well as positive and negative controls;

FIG. 14 is a graphical illustration showing the reduction of tenocytes in the media for the various wells over time (Ex. 2 and 3: Comp. Ex. 2 and 3), a greater reduction of tenocytes in the media is indicative of the tenocytes better impregnating the scaffold;

FIG. 15a shows laser scanning confocal microscopic images of a plan view of the COL/PLA scaffold (Ex. 2) and PLA scaffold (Comp. Ex. 2) wherein the blue dots represent tenocytes at day 1 and day 7 after seeding.

FIG. 15b shows laser scanning confocal microscopic images of a z-direction (side view) of the COL/PLA scaffold (Ex. 2) and PLA scaffold (Comp. Ex. 2) wherein the blue dots represent tenocytes at day 14.

### DETAILED DESCRIPTION OF THE INVENTION

According to an embodiment of the present invention, a fiber-based scaffold for multiple tissue junction regeneration comprises biological collagen fibers and resorbable synthetic fibers. When the scaffold is attached to a musculoskeletal system in a host, the synthetic fibers remain in the host for a period ranging from 6 weeks to 3 years.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “and/or”, when used in a list of two or more items, means that any one of the listed items can be employed by itself, or any combination of two or more of the listed items can be employed. For example, if a composition is described as containing compounds A, B, “and/or” C, the composition may contain A alone; B alone; C alone; A and B in combination; A and C in combination; B and C in combination; or A, B, and C in combination.

As used herein, the term “musculoskeletal system”, refers to bones, muscles, cartilage, tendons, ligaments, joints, and other connective tissue of a body. The attachment of the scaffold to a site on the musculoskeletal system is typically referring to the musculoskeletal system of a human, whereas the collagen fibers may comprise collagen collected from the musculoskeletal system of species other than human.

The shape and size of the scaffold is not particularly limited and can be readily customized for different procedures and different hosts. Non-limiting examples of shape include square, rectangle, oval, or circular. In some embodiments, the scaffold is in the shape of an essentially linear ribbon, or a ribbon that is Y-shaped, or a ribbon that is X-shaped, or a ribbon that is V-shaped. The thickness of the scaffold is also not particularly limited, so long as it is thick enough to provide the necessary mechanical strength required for the repair, and small enough to readily resorb in the appropriate amount of time. In some embodiments, the scaffold is a cylindrical shape and suited for arthroscopic or minimally-invasive delivery of the scaffold.

In some embodiments, the synthetic fibers have cross-sectional shapes selected from the group including round, 4DG, and trilobal. An example of 4DG fibers can be found in U.S. Pat. No. 8,129,019 B2.

The manner in which the scaffold is secured to the musculoskeletal structure of the host is not particularly limiting. Commonly used suture anchor designs and techniques can be used, such as, for example, in the transosseous-equivalent (TOE) rotator cuff repair technique.

In some embodiments, the scaffold can be applied to reconstruction or augmentation of the rotator cuff repair or augmentation. Additional non-limiting embodiments of the scaffold application include knee extensor mechanism reconstruction (e.g., quadriceps or patellar tendon reconstruction or augmentation with primary repair), Achilles tendon reconstruction or augmentation with primary repair, pectoralis major tendon augmentation with primary repair, or augmentation in tendon transfers around the shoulder for rotator cuff deficiency (e.g., lower trapezius muscle transfer or latissimus dorsi/teres major transfer). Further non-limiting applications of the scaffold include areas of ligament reconstruction, specifically extra-articular ligament reconstruction/augmentation of the knee or elbow (e.g., medial collateral ligament reconstruction).

Item 1—A fiber-based scaffold for multiple tissue junction regeneration comprising: a) biological collagen fibers, and b) resorbable synthetic fibers, wherein, when the scaffold is attached to a musculoskeletal system in a host, the synthetic fibers remain in the host for a period ranging from 6 weeks to 3 years.

Item 2—The scaffold of item 1, wherein the collagen fibers are electrochemically aligned fibers and wherein an amount of the collagen fibers ranges from 25 wt. % to 35 wt. % on a total fiber weight basis.

Item 3—The scaffold of any of items 1 or 2, wherein the collagen fibers comprise a collagen from a species selected from the group consisting of rat, pig, and human.

Item 4—The scaffold of item 3, wherein the collagen was harvested from a site of a musculoskeletal system selected from the group consisting of rotator cuff, acromioclavicular, Achilles tendon, pectoralis major tendon, lower trapezius muscle, latissimus dorsi/teres major, and/or medial collateral ligament.

Item 5—The scaffold of any of items 1-4, wherein the synthetic fibers comprise poly(lactic) acid (PLA), poly(glycolic) acid (PGA), poly(lactic-co-glycolic acid) (PLGA), polycaprolactone (PCL), and/or polydioxanone (PDO); or wherein the synthetic fibers comprise poly(lactic) acid (PLA).

Item 6—The scaffold of any of items 1-5, wherein the synthetic fibers have cross-sectional shapes selected from the group consisting of round, 4DG, and trilobal.

Item 7—The scaffold of any of items 1-6, wherein the synthetic fibers remain in the host for a period ranging from 8 weeks to 3 years, or 12 weeks to 3 years, or 6 months to 3 years, or 8 weeks to 2 years, or 12 weeks to 2 years, or 6 months to 2 years, or 8 weeks to 18 months, or 12 weeks to 18 months, or 6 months to 18 months.

Item 8—The scaffold of any of items 1-7, wherein at least one physical property of the scaffold, selected from the group consisting of maximum stress and/or elastic modulus, falls within the range of values for native rotator cuff tendons; or wherein at least one physical property of the scaffold, selected from the group consisting of maximum stress and/or elastic modulus, falls within the range of values for the musculoskeletal system site selected from the group consisting of rotator cuff, acromioclavicular, Achilles tendon, pectoralis major tendon, lower trapezius muscle, latissimus dorsi/teres major, and/or medial collateral ligament.

Item 9—The scaffold of any of items 1-8, wherein the collagen fibers have a diameter ranging from 0.1 mm to 10 mm and are formed into collagen yarns have a total denier ranging from 100 to 1,000; or wherein the collagen fibers have a diameter ranging from 0.1 mm to 10 mm and are formed into collagen yarns have a total denier ranging from 100 to 500.

Item 10—The scaffold of any of items 1-9, wherein the synthetic fibers have a denier per filament ranging from 0.5 to 10 and are formed into synthetic yarns having a total denier ranging from 50 to 500; or wherein the synthetic fibers have a denier per filament ranging from 1 to 8 and are formed into synthetic yarns having a total denier ranging from 50 to 300.

Item 11—The scaffold of item 10, wherein the scaffold is a woven ribbon, wherein a weft direction comprises the synthetic yarns and a warp direction comprises plied yarns comprising at least one of the synthetic yarns and at least one of the collagen yarns.

Item 12—The scaffold of item 11, wherein the woven ribbon is essentially linear in shape, or wherein the woven ribbon is Y-shaped, or wherein the woven ribbon is X-shaped, or wherein the woven ribbon is V-shaped.

Item 13—The scaffold of item 10, wherein the scaffold comprises braids of the collagen yarns, the synthetic yarns, and/or plied yarns comprising at least one of the synthetic yarns and at least one of the collagen yarns.

Item 14—The scaffold of any of items 1-13, wherein the scaffold comprises a structural gradient whereby mechanical properties vary along a direction of the scaffold.

Item 15—A method for making the fiber-based scaffold for multiple tissue junction regeneration of any of items 1-8, the method comprising: a) obtaining biological collagen fibers; b) obtaining resorbable synthetic fibers; and c) fabricating the collagen fibers and the synthetic fibers into the scaffold.

Item 16—The method of item 15, wherein the collagen fibers have a diameter ranging from 0.1 mm to 10 mm and are formed into collagen yarns have a total denier ranging from 100 to 1,000; or wherein the collagen fibers have a diameter ranging from 0.1 mm to 10 mm and are formed into collagen yarns have a total denier ranging from 100 to 500.

Item 17—The method of any of items 15 or 16, wherein the synthetic fibers have a denier per filament ranging from 0.5 to 10 and are formed into synthetic yarns having a total denier ranging from 50 to 500; or wherein the synthetic fibers have a denier per filament ranging from 1 to 8 and are formed into synthetic yarns having a total denier ranging from 50 to 300.

Item 18—The method of item 17, wherein the fabricating comprises weaving a ribbon or other shape, wherein a weft direction comprises the synthetic yarns and wherein a warp direction comprises plied yarns comprising at least one of the synthetic yarns and at least one of the collagen yarns.

Item 19—The method of item 18, wherein the spacing between the synthetic yarns in the weft direction varies, whereby a pore size ranges from 30 μm to 300 μm, and the scaffold exhibits a structural gradient and mechanical properties vary along a direction of the scaffold.

Item 20—The method of item 17, wherein the fabricating comprises braiding the collagen yarns, the synthetic yarns, and/or plied yarns comprising at least one of the synthetic yarns and at least one of the collagen yarns wherein a number of bobbins varies from 8 to 24, a picks per inch varies from 12 to 84, and a poor size ranges from 0.5 to 340 μm; or wherein the fabricating comprises braiding the collagen yarns, the synthetic yarns, and/or plied yarns comprising at least one of the synthetic yarns and at least one of the collagen yarns wherein a number of bobbins varies from 8 to 24, a picks per inch varies from 12 to 64, and a poor size ranges from 1 to 250 μm.

Item 21—A method for attaching the fiber-based scaffold of any of items 1-14 for multiple tissue junction regeneration to a host, the method comprising: a) obtaining the scaffold; b) securing a first section of the scaffold to a first site of the musculoskeletal system of the host; and c) securing a second section of the scaffold to a second site of the musculoskeletal system of the host, wherein the second section is distal from the first section, wherein the first site and the second site are joined in tension by the scaffold.

Item 22—The method of item 21, wherein the first site is selected from the group consisting of bones, ligaments, joints, and tendons and wherein the second site is selected from the group consisting of bones, ligaments, joints, and tendons.

Item 23—The method of any of items 21 or 22, wherein the securing of the first section to the first site and the second section to the second site are independently selected from the group consisting of suture anchors, medial row anchors, and/or suture tape.

Item 24—The method of any of items 21-23, wherein a surgical procedure is selected from the group consisting of a rotator cuff repair or augmentation; a knee extensor mechanism reconstruction, an Achilles tendon reconstruction or augmentation with primary repair; a pectoralis major tendon augmentation with primary repair; an augmentation in tendon transfers around the shoulder for rotator cuff deficiency (e.g., lower trapezius muscle transfer or latissimus dorsi/teres major transfer); an extra-articular reconstruction or augmentation of the knee; and an extra-articular reconstruction or augmentation of the elbow, and wherein the first site and the second site correspond to the surgical procedure.

Item 25—The method of item 24, wherein the surgical procedure consists of the rotator cuff repair or augmentation and the first site consists of a glenoid and/or a tendon attached to the glenoid and the second site consists of a humerus.

Item 26—The method of item 25, wherein the first section of the scaffold is secured to the glenoid and/or a tendon attached to the glenoid using at least two of the suture anchors, and wherein the second section of the scaffold is secured to the humerus using two of the medial row anchors positional just lateral to an articular margin and the suture tape.

Item 27—The method of item 26, wherein the at least two suture anchors are selected from the group consisting of Q-FIX and Osteoraptors, and the suture tape comprises Ultratape.

Item 28—The method of any of items 25-27, further comprising using two lateral row anchors to compress a lateral aspect of the scaffold to a lateral footprint in a transosseous-equivalent (TOE) rotator cuff repair construct.

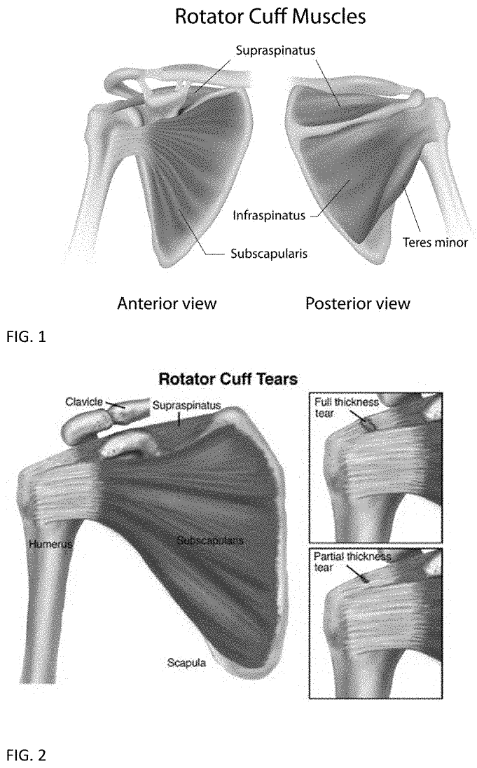

FIG. 1 shows an anterior view and a posterior view of rotator cuff muscles. Identified muscles include the supraspinatus, infraspinatus, subscapularis, and teres minor. FIG. 2 shows a rotator cuff and two enlarged schematics showing a full thickness tear and a partial thickness tear.

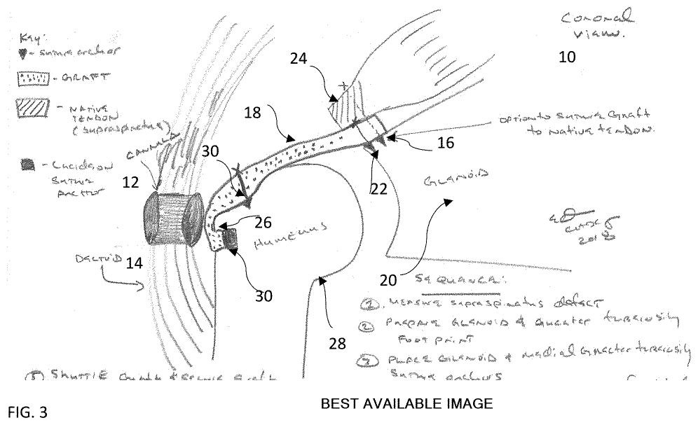

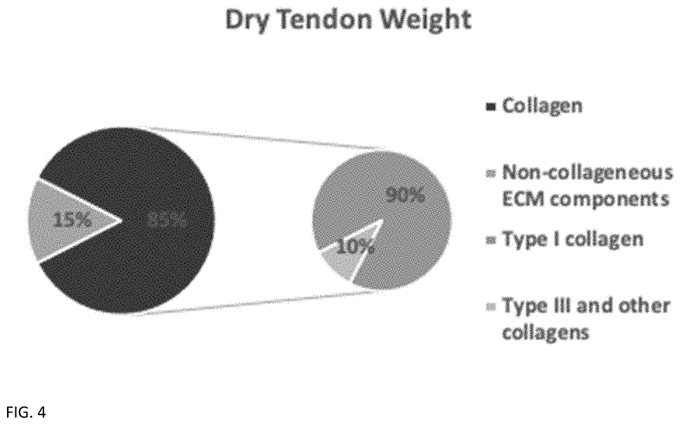

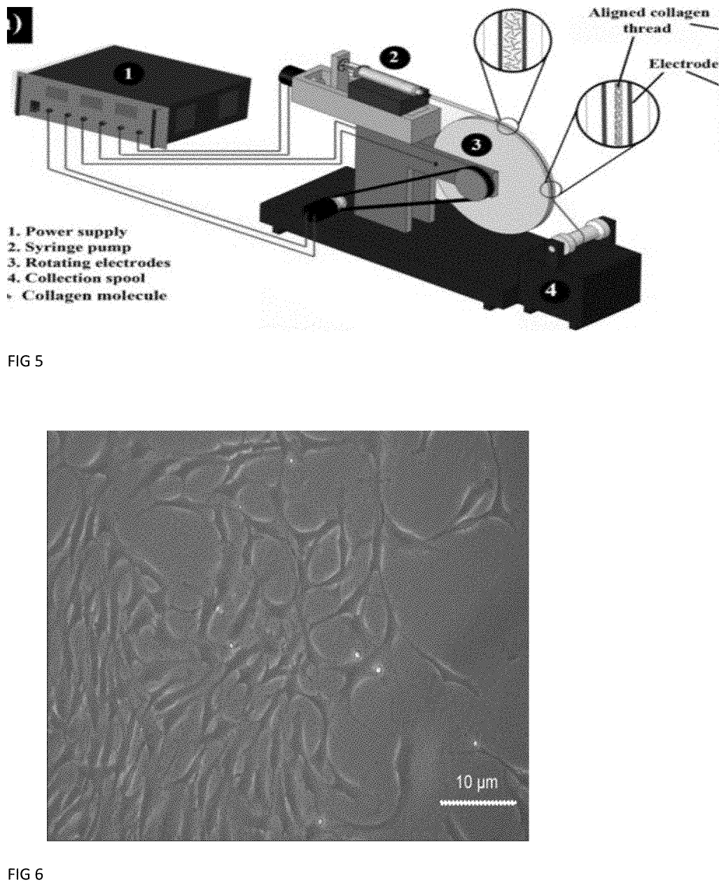

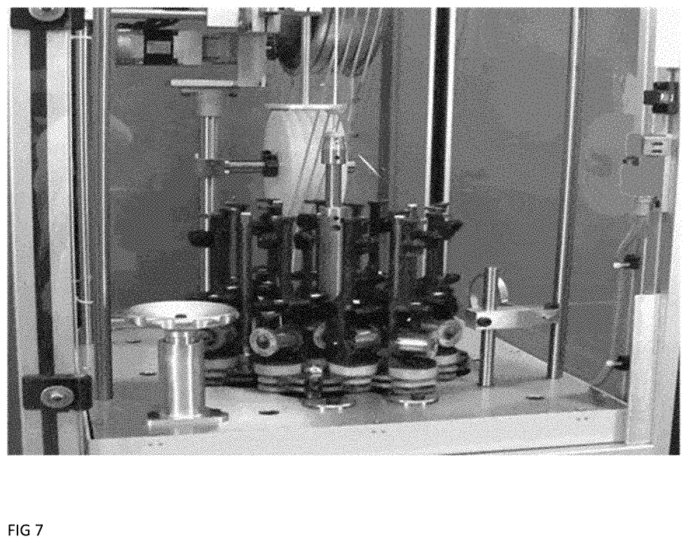

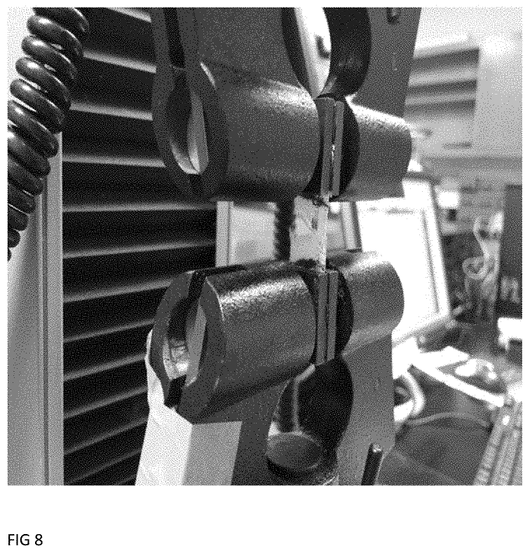

FIG. 3 is a schematic of a coronal view 10 of a nonlimiting embodiment of the present invention. The illustrated graft delivery uses routine graft-passing arthroscopic techniques using cannula 12 to pass through deltoid 14. The first end 16 of scaffold 18 can be secured to the glenoid 20 using two or more suture anchors 22 or can be sutured to native tendon 24. The second end 26 of the scaffold 18 can be secured to the humerus 28 using transosseous-equivalent (TOE) rotator cuff repair techniques with appropriate tension using anchor 30. An additional anchor 32 secures scaffold 18 to the humerus.

### EXAMPLES

### Example 1 and Comparative Example 1

To make the scaffold of Example 1, two collagen yarns where plied together and two PLA yarns, each having 72 filaments and 146 denier, were plied together. The collagen yarns were spun at Dr. Ozan Akkus's laboratory at Case Western Reserve University (See, US Pub. No. 2018/0312988 A1, Nov. 1, 2018). The plied collagen yarn and the plied PLA yarn were then plied together to form a COL/PLA yarn. The scaffold was constructed as a ribbon fabric with the COL/PLA yarns in the weft direction and 100% PLA yarns in the warp direction. The scaffold had 28 warp ends which fabricated an approximately 17 mm side ribbon with pore sizes between 60 μm and 160 μm.

The scaffold of Comparative Example 1 was made in a similar manner, except that 100% PLA yarns were used in both the weft and the warp direction.

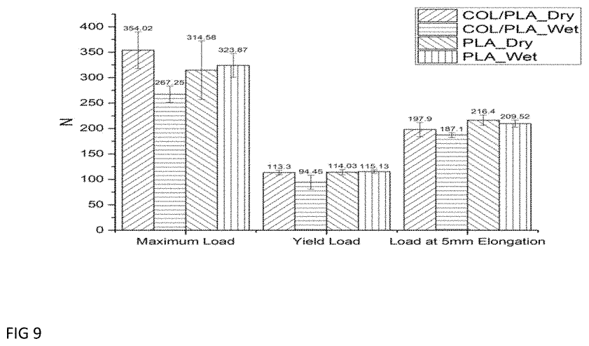

The ultimate tensile properties of the scaffolds were tested under both wet and dry conditions on an Instron Model 5584 mechanical tester (Norwood, Mass., USA) with a 10 mm gauge length and a crosshead speed of 30 mm/min. The wet condition was achieved by pretreating the samples in 0.01M Phosphate Buffered Saline (PBS) for two hours before testing. The maximum load, yield load, and load at 5 mm elongation are shown for Example 1 (COL/PLA scaffold) and Comparative Example 1 (PLA scaffold) under dry and wet conditions in FIG. 9.

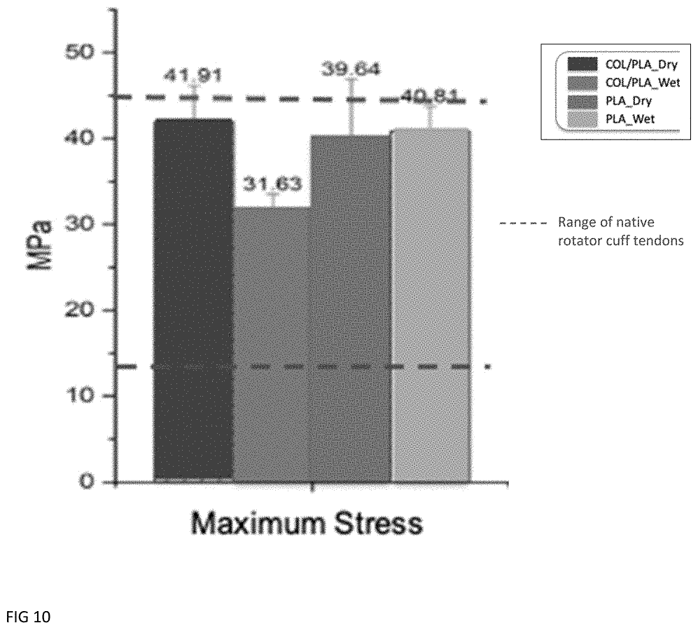

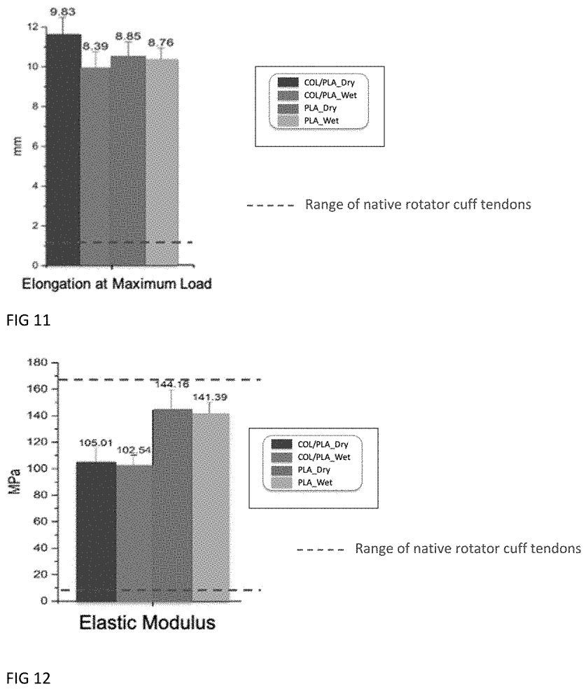

Additional mechanical tests were performed on Example 1 and Comparative Example 1 under both dry and wet conditions (i.e., pretreated in PBS for two hours). Results are shown in FIGS. 10-12 along with the typical range for rotator cuff tendons. FIG. 10 shows the maximum stress, FIG. 11 shows the elongation at maximum load, and FIG. 12 shows the elastic modulus of each scaffold under dry and wet conditions. The maximum stress of the COL/PLA scaffold in a dry condition was significantly higher than when wet, the PLA scaffold had an equivalent maximum stress when tested under dry and wet conditions with no significant difference between the two, there is also no statistical difference between the dry COL/PLA scaffold and the dry PLA scaffold. The only significant difference for the elongation at maximum load was found between the COL/PLA scaffold tested in a dry compared to a wet condition. The elastic modulus is calculated from the slope of the linear region of stress-strain curve, the experimental results indicated that both dry and wet PLA scaffolds had a significantly higher elastic modulus than those of the COL/PLA scaffolds. Although the presence of collagen yarns within the structure has effects on the tensile properties of the COL/PLA scaffold (Example 1), the maximum stress, elongation at break, and elastic modulus are still within the required range for an augmentation scaffold suitable for rotator cuff repair.

### Examples 2 and 3 and Comparative Examples 2 and 3

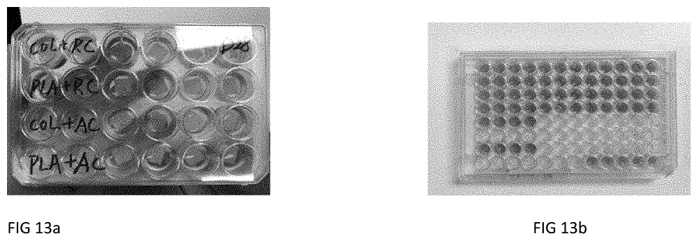

For Example 2, six sets of 1 cm×1 cm COL/PLA scaffolds (Ex. 1) were seeded with ten to the fourth rat rotator cuff tenocytes in a media (COL/PLA-RC). For Comparative Example 2, six sets of 1 cm×1 cm PLA scaffolds (Comp. Ex. 1) were seeded with ten to the fourth rat rotator cuff tenocytes in a media (PLA-RC). For Example 3, six sets of 1 cm×1 cm COL/PLA scaffolds (Ex. 1) were seeded with ten to the fourth rat acromioclavicular joint tenocytes in a media (COL/PLA-AC). For Comparative Example 3, six sets of 1 cm×1 cm PLA scaffolds (Comp. Ex. 1) were seeded with ten to the fourth rat acromioclavicular joint tenocytes in a media (PLA-AC). The 24-well plate with Examples 2 and 3 and Comparative Examples 2 and 3 is shown in FIG. 13a.

In order to readily infer the proliferation of tenocytes on the scaffolds, the media was tested using 10% ALARMARBLUE at 1, 7, 14, and 28 days. 100 μL media for each set of Examples 2 and 3 and Comparative Examples 2 and 3 was harvested and tested on days 1, 7, 14, and 28. These samples along with both a positive and negative controls are shown in FIG. 13b. The positive control was autoclaved 10% ALARMARBLUE in media and the negative control was 10% ALARMARBLUE in media. The fluorescence measurement wavelengths were 550 nm for excitation and 590 nm for emission. The percentage reduction in ALARMARBLUE (%) was calculated as follows:

$$Percentage Reduction of Alamar Blue ( \% ) = \frac{Fluorescence of Specimen - Fluoresence of negative control}{\begin{matrix}Fluorescence of positive control - \\ Fluorescence of negative control\end{matrix}} \times 100$$

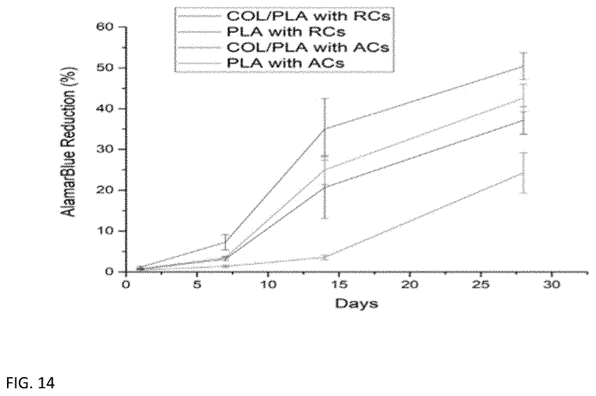

The average results for the percent reduction in ALARMARBLUE for Examples 2 and 3 and Comparative Examples 2 and 3 are shown in FIG. 14. For both the rat rotator cuff tenocytes and the acromioclavicular joint tenocytes, the COL/PLA samples showed a statistically significant higher reduction in tenocytes in the media, which implies better proliferation of tenocytes on the COL/PLA scaffolds (Ex. 2 and 3), than the PLA scaffolds (Comp. Ex. 2 and 3).

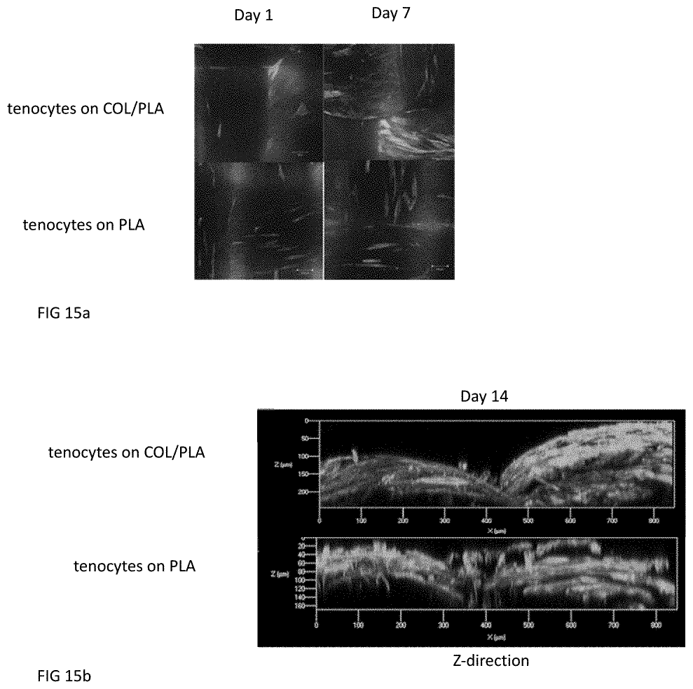

Confocal microscopic images for rotator cuff tenocytes on COL/PLA-RC scaffold (Ex. 2) and pure PLA-RC scaffold (Comp. Ex. 2) on day 1, day 7, and day 14 are shown in FIGS. 15a and 15b. The blue dots are the cell nuclei stained by 4′,6-diamidino-2-phenylindole (DAPI). FIGS. 15a and 15b show more blue dots on COL/PLA-RC scaffolds (EX. 2) on day 7 and day 14, indicating that more cells were attached to the COL/PLA scaffold, and confirming that tenocytes preferred to proliferate on COL/PLA scaffold as compared to the PLA scaffold.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A fiber-based scaffold for multiple tissue junction regeneration comprising:
a) biological collagen fibers; and
b) resorbable synthetic fibers,
wherein, when the scaffold is attached to a musculoskeletal system in a host, the synthetic fibers remain in the host for a period ranging from 6 weeks to 3 years.

2. The scaffold of claim 1, wherein the collagen fibers are electrochemically aligned fibers and wherein an amount of the collagen fibers ranges from 25 wt. % to 35 wt. % on a total fiber weight basis.

3. The scaffold of claim 1, wherein the collagen fibers comprise a collagen from a species selected from the group consisting of rat, pig, and human, and wherein the collagen was harvested from a site of a musculoskeletal system selected from the group consisting of rotator cuff, acromioclavicular, Achilles tendon, pectoralis major tendon, lower trapezius muscle, latissimus dorsi/teres major, and/or medial collateral ligament.

4. The scaffold of claim 1, wherein the synthetic fibers comprise poly(lactic) acid (PLA), poly(glycolic) acid (PGA), poly(lactic-co-glycolic acid) (PLGA), polycaprolactone (PCL), and/or polydioxanone (PDO); or wherein the synthetic fibers comprise poly(lactic) acid (PLA).

5. The scaffold of claim 1 wherein at least one physical property of the scaffold, selected from the group consisting of maximum stress and/or elastic modulus, falls within the range of values for the musculoskeletal system site selected from the group consisting of rotator cuff, acromioclavicular, Achilles tendon, pectoralis major tendon, lower trapezius muscle, latissimus dorsi/teres major, and/or medial collateral ligament

6. The scaffold of claim 1, wherein the collagen fibers have a diameter ranging from 0.1 mm to 10 mm and are formed into collagen yarns have a total denier ranging from 100 to 1,000, and wherein the synthetic fibers have a denier per filament ranging from 0.5 to 10 and are formed into synthetic yarns having a total denier ranging from 50 to 500.

7. The scaffold of claim 6, wherein the scaffold is a woven ribbon, wherein a weft direction comprises the synthetic yarns and a warp direction comprises plied yarns comprising at least one of the synthetic yarns and at least one of the collagen yarns.

8. The scaffold of claim 1, wherein the scaffold comprises a structural gradient whereby mechanical properties vary along a direction of the scaffold.

9. A method for making a fiber-based scaffold for multiple tissue junction regeneration, the method comprising:
a) obtaining biological collagen fibers;
b) obtaining resorbable synthetic fibers; and
c) fabricating the collagen fibers and the synthetic fibers into the scaffold,
wherein, when the scaffold is attached to a musculoskeletal system in a host, the synthetic fibers remain in the host for a period ranging from 6 weeks to 3 years.

10. The method of claim 9, wherein the collagen fibers have a diameter ranging from 0.1 mm to 10 mm and are formed into collagen yarns have a total denier ranging from 100 to 1,000 and wherein the synthetic fibers have a denier per filament ranging from 0.5 to 10 and are formed into synthetic yarns having a total denier ranging from 50 to 500.

11. The method of claim 9, wherein the fabricating comprises weaving a ribbon or other shape, wherein a weft direction comprises the synthetic yarns and wherein a warp direction comprises plied yarns comprising at least one of the synthetic yarns and at least one of the collagen yarns.

12. The method of claim 11, wherein the spacing between the synthetic yarns in the weft direction varies, whereby a pore size ranges from 30 μm to 300 μm, and the scaffold exhibits a structural gradient and mechanical properties vary along a direction of the scaffold.

13. The method of claim 11, wherein the fabricating comprises braiding the collagen yarns, the synthetic yarns, and/or plied yarns comprising at least one of the synthetic yarns and at least one of the collagen yarns wherein a number of bobbins varies from 8 to 24, a picks per inch varies from 12 to 84, and a poor size ranges from 0.5 to 340 μm.

14. A method for attaching a fiber-based scaffold for multiple tissue junction regeneration to a host, the method comprising:
a) obtaining the scaffold;
b) securing a first section of the scaffold to a first site of the musculoskeletal system of the host; and
c) securing a second section of the scaffold to a second site of the musculoskeletal system of the host, wherein the second section is distal from the first section,
wherein the first site and the second site are joined in tension by the scaffold,
wherein, when the scaffold is attached to a musculoskeletal system in a host, the synthetic fibers remain in the host for a period ranging from 6 weeks to 3 years,
wherein the scaffold is a woven ribbon, wherein a weft direction comprises the synthetic yarns and a warp direction comprises plied yarns comprising at least one of the synthetic yarns and at least one of the collagen yarns, and
wherein the ribbon is in a shape selected from the group consisting of rectangular, Y-shaped, X-shaped, and V-shaped.

15. The method of 14, wherein the collagen fibers have a diameter ranging from 0.1 mm to 10 mm and are formed into collagen yarns have a total denier ranging from 100 to 1,000, and wherein the synthetic fibers have a denier per filament ranging from 0.5 to 10 and are formed into synthetic yarns having a total denier ranging from 50 to 500.

16. The method of claim 14, wherein the first site is selected from the group consisting of bones, ligaments, joints, and tendons and wherein the second site is selected from the group consisting of bones, ligaments, joints, and tendons.

17. The method of claim 14, wherein the securing of the first section to the first site and the second section to the second site are independently selected from the group consisting of suture anchors, medial row anchors, and/or suture tape.

18. The method of claim 14, wherein a surgical procedure is selected from the group consisting of a rotator cuff repair or augmentation; a knee extensor mechanism reconstruction, an Achilles tendon reconstruction or augmentation with primary repair; a pectoralis major tendon augmentation with primary repair; an augmentation in tendon transfers around the shoulder for rotator cuff deficiency (e.g., lower trapezius muscle transfer or latissimus dorsi/teres major transfer); an extra-articular reconstruction or augmentation of the knee; and an extra-articular reconstruction or augmentation of the elbow, and wherein the first site and the second site correspond to the surgical procedure.

19. The method of claim 18, wherein the surgical procedure consists of the rotator cuff repair or augmentation and the first site consists of a glenoid and/or a tendon attached to the glenoid and the second site consists of a humerus.

20. The method of claim 19, wherein the first section of the scaffold is secured to the glenoid and/or a tendon attached to the glenoid using at least two of the suture anchors, wherein the second section of the scaffold is secured to the humerus using two of the medial row anchors positional just lateral to an articular margin and the suture tape, wherein the at least two suture anchors are selected from the group consisting of Q-FIX and Osteoraptors, and the suture tape comprises Ultratape, and
further comprising using two lateral row anchors to compress a lateral aspect of the scaffold to a lateral footprint in a transosseous-equivalent (TOE) rotator cuff repair construct.
